(19) United States
(12) Patent Application Publication
(10) Pub. No.: US 2023/0227888 A1
(43) Pub. Date: Jul. 20, 2023

(54) POINT OF NEED DIAGNOSTIC DEVICE AND METHODS OF USE THEREOF

(71) Applicant: GoDx, Inc., Madison, WI (US)

(72) Inventors: Chang Hee KIM, Waunakee, WI (US); Jacob Jerome TOKAR, Madison, WI (US); Xiao JIANG, Madison, WI (US)

(21) Appl. No.: 17/925,223

(22) PCT Filed: May 12, 2021
(86) PCT No.: PCT/US2021/032039

§ 371 Date: Nov. 14, 2022

Related U.S. Application Data
(60) Provisional application No. 63/023,652, filed on May 12, 2020.

(51) Int. Cl. C12Q 1/68; B01L 3/00

(52) U.S. Cl. CPC C12Q 1/68; B01L 3/502715; B01L 3/5023; B01L 2200/0621; B01L 2300/087; B01L 2300/18; B01L 2400/043; B01L 2300/0627; B01L 2200/16; B01L 2300/025; B01L 2300/0681

(57) ABSTRACT

The present invention provides point-of-need diagnostic devices and kits for detecting a target nucleic acid sequence in a sample. Methods of using the point-of-need diagnostic devices or the kits disclosed are also provided.

### CROSS-REFERENCE TO RELATED APPLICATIONS

This application priority to U.S. Provisional Application No. 63/023,652 filed on May 12, 2020, the contents of which are incorporated by reference in their entireties.

### STATEMENT REGARDING FEDERALLY SPONSORED RESEARCH

This invention was made with government support under R44TR001912 awarded by National Institutes of Health. The government has certain rights in the invention.

### INTRODUCTION

Point-of-need testing is intended to bring diagnostic testing conveniently and immediately to the patient, at the time and place of patient care and/or need. To meet the requirements set forth by the World Health Organization, such tests must be low cost, portable, and not require plug-in electricity for storage or operation. Further, they must be completed in less than four user steps, none of which can involve volumetric measurement, time-dependent actions, or physical difficulty. Ideally, the clinical care team receives the results of point-of-need tests rapidly, allowing more immediate clinical management decisions to be made.

Several recent advances have improved our ability to detect target nucleic acids using a point-of-need device. These advances include hollow-centered silica microspheres that allow for rapid separation of nucleic acids form complex biological samples (International Patent Pub. No. WO/2019/109092) and lateral flow devices that allow for multiplexed detection of target nucleic acids (U.S. Patent Publication No. 2018/0148774). However, there remains an unmet need in the art for self-contained diagnostic devices that detect target nucleic acids and that meet point-of-need standards.

### SUMMARY

The present invention provides point-of-need diagnostic devices for detecting a target nucleic acid sequence in a sample. The devices comprise a housing with a sample inlet for receiving the sample. The housing contains the following within it: (a) a nucleic acid binding stage that is optionally movable comprising a permeable nucleic acid binding substrate and an eluate outlet by which an eluate may exit the binding stage; (b) a wash reservoir containing a wash buffer; (c) an elution reservoir containing an elution buffer; (d) a moveable or stationary amplification stage comprising an eluate inlet for receiving the eluate from the nucleic acid binding stage, at least one reaction chamber comprising a nucleic acid amplification reagent, and an amplicon outlet by which an amplified sample may exit the amplification stage; (e) a running buffer reservoir containing a running buffer; and (f) a detection device that provides a readout that indicates whether the target nucleic acid is present in the sample. The nucleic acid binding stage may be configured such that it can be positioned to be in fluid communication with the sample inlet, the wash reservoir, the elution reservoir, and the amplification stage via the eluate outlet. Additionally, the amplification stage may be configured such that it can be positioned to be in fluid communication with the running buffer reservoir and the detection device via the amplicon outlet. In some embodiments, the nucleic acid binding stage is configured to move in response to a mechanical force or a modulation in a magnetic potential or an electric potential; comprises a sponge ramp; comprises a permeable nucleic acid binding substrate comprises laminated silica microspheres; or any combination thereof. In some embodiments, the device optionally comprises one or more valves for controlling the migration of sample through the device. In some embodiments, the wash reservoir is optionally in fluid communication with a wash sponge or wash hopper. In some embodiments, the elution reservoir is optionally in fluid communication with an elution sponge or elution hopper. In some embodiments, the housing is configured to interface with a lysis cartridge and/or a controller.

In a second aspect, the present invention provides kits comprising the point-of-need diagnostic devices disclosed herein and further comprising one or more of a controller, a lysis cartridge, a lysis agent, a collection device, and a heating element.

In a third aspect, the present invention provides methods of using the point-of-need diagnostic devices or the kits disclosed herein to detect a target nucleic acid sequence in a sample. The methods involve (a) loading a sample lysate into the diagnostic device via the sample inlet, thereby contacting the sample with nucleic acid binding substrate; (b) contacting the nucleic acid binding substrate with the wash buffer, wherein the contacting step optionally comprises positioning the nucleic acid binding stage to be in fluid communication with the wash reservoir; (c) contacting the nucleic acid binding substrate with the elution buffer and eluting the eluate, wherein the contacting step optionally comprises positioning the nucleic acid binding stage to be in fluid communication with the elution reservoir and the amplification stage; (d) heating the eluate and the nucleic acid amplification reagent in the reaction chamber under conditions sufficient for amplifying the target nucleic acid, thereby preparing the amplified sample; (e) transferring the amplified sample to the diagnostic device, wherein the transferring step optionally comprises positioning the amplification stage to be in fluid communication with both the running buffer reservoir and the detection device or actuating a valve positioned between the amplification stage and the diagnostic device; and (f) inspecting the readout provided by the detection device to determine whether the target nucleic acid is present in the sample.

In some embodiments, the device further comprises the controller. The controller may be configured to interface with the housing and provide the mechanical force or modulate the magnetic potential or the electric potential to move the moveable nucleic acid binding stage into or out of fluid communication with the sample inlet, the wash reservoir, the elution reservoir, and the amplification stage or actuate the one of more valves for controlling the migration of sample through the device at predetermined times. In some embodiments, the controller comprises a movable magnet; a microcontroller configured to execute a set of instructions for moving the moveable nucleic acid binding stage into or out of fluid communication with the sample inlet, the wash reservoir, the elution reservoir, and the amplification stage or actuating the one or more valves for controlling the migration of sample through the device, a heating element, a power source, a readout device, or any combination thereof.

In some embodiments, the device further comprises a lysis cartridge. The lysis cartridge may comprise a filter configured to separate particulates from a liquid lysis sample and a sample evacuation device. In some embodiments, the lysis cartridge comprises a filter membrane or a porous foam filter; a chemical contaminant sequestration material; a manually or automatically actuated plunger; is incapable of evacuating a sample from the lysis cartridge unless a fluid connection is formed between with a sample inlet of a diagnostic device and the lysis cartridge, or any combination thereof.

### BRIEF DESCRIPTION OF THE DRAWINGS

FIG. 1 shows a schematic depicting an exemplary point-of-need diagnostic device. This design includes a lysis cartridge that is used to lyse the sample, forming a lysate solution. The nucleic acid binding stage (Slider 1) is initially positioned directly beneath the sample inlet so it can collect and bind nucleic acids from the lysate solution. The lysate seeps through a nucleic acid binding substrate positioned at the bottom of a funnel built into Slider 1. The substrate binds nucleic acids and filters out large particulate. The user then slides Slider 1 from position one—beneath the sample inlet—to position two. This action may pierce a blister pouch holding wash buffer, which flows through the substrate of Slider 1, washing the bound nucleic acids. A waste pad beneath Slider 1 absorbs the lysis and wash buffers passing through the nucleic acid binding substrate during both of these initial steps. Next, Slider 1 is slid from position two to position three where it may pierce a blister pouch containing elution buffer. In position three, Slider 1 is positioned over the eluate inlet of the amplification stage (Slider 2) in such a way that when the elution buffer flows through the substrate of Slider 1, it delivers the eluate to the eluate inlet of Slider 2. The eluate then flows through microchannels to the reaction chambers of Slider 2, which contain a nucleic acid amplification reagent. In this embodiment, Slider 2 comprises four reaction chambers: three of the reaction chambers contain the necessary enzymes and primers to amplify diagnostic markers that are indicative of the presence of a pathogen, and the fourth chamber contains primers to amplify a control, such as human ActB or RNAseP. Amplification is allowed to proceed for approximately 15-20 minutes. Then, the user moves Slider 2 from its initial position to its final position where Slider 2 may pierce a blister pouch containing lateral flow assay (LFA) running buffer. The running buffer forces the amplicons to flow from the reaction chambers through microchannels that converge at the output port of Slider 2, positioned directly beneath the collection pad of the diagnostic device (LFA). When the output droplet merges with the LFA pad, capillary flow within the LFA drives further flow of the amplicons from Slider 2 through the LFA for pathogen detection.

FIG. 2 shows a schematic depicting an exemplary point-of-need kit. The platform consists of a sample collection tube, a transfer pipette or swab, a lysis cartridge, a disposable processing/detection device, and a reusable handheld controller.

FIG. 3 shows a schematic depicting an exemplary lysis cartridge. The lysis cartridge may be configured to filter out particulate and chemical inhibitors to improve nucleic acid recovery and amplification.

FIG. 4 shows a schematic depicting an exemplary nucleic acid binding stage. The stage consists of a collection funnel, an outlet where the nucleic acid binding substrate is affixed, and a sponge ramp that compresses and drains reagent sponges.

FIG. 5 shows a schematic depicting an exemplary nucleic acid purification device that utilizes laminated glass microspheres. (a): Schematic diagram of the testing design and lamination process. The lamination process is performed by sandwiching the microspheres between two glass fiber membranes. The glass fiber membrane-microspheres sandwich is then sandwiched between two pieces of thermoplastic film. The final layered assembly is laminated by passing through a heat laminator. The laminated microspheres are adhered to the bottom of the 3D-printed funnel for testing nucleic acid isolation. (b): A 3D-printed funnel with laminated borosilicate glass microspheres attached to the opening via a piece of double-sided adhesive. (c): A photograph demonstrating analyte filtration using food dye.

FIG. 6 shows a schematic depicting reagent delivery using sponges. When a sample is being processed, the shelf-life reagent reservoirs are pierced and drained into sponges for temporary storage as they await their turn in the protocol sequence. The sponge ramp gently compresses the sponges, draining reagents into the funnel of the binding stage.

FIG. 7 shows a schematic depicting an exemplary protocol in which the handheld device is used to analyze a sample. (a): Sample is lysed in the lysis cartridge, which is a pierce-bottom tube. (b): The bottom of the lysis cartridge is pierced as it is inserted into the device, draining the lysate into the processing funnel. The binding membrane attached to the funnel output binds nucleic acids, such as DNA, as the lysate drains through. Wash and elution buffers are subsequently drained through the membrane, with the eluate wicking into a microchannel (paper or capillary) rather than the waste pad. (c): Eluate wicks through the microchannel into the reaction chamber. (d): Nucleic acids, such as DNA, and reaction reagents are mixed when rehydration buffer is added to the chamber. (e): An amplification reaction begins when the chamber is heated. (f): Amplicons are wicked through another microchannel into the sample pad. (g): Running buffer is released from a reagent blister pouch, driving amplicons through to the LFA beyond.

FIG. 8 shows a graph in which human RNA recovered from laminated borosilicate glass microspheres is quantified. Recovery from laminated borosilicate glass microspheres fabricated using two techniques (i.e., PVA bead and pipette bead) is compared to recovery from a laminated glass fiber pad. The RNA recovery rate is indicated on the left vertical axis and the time required to filter the lysis buffer is indicated on the right vertical axis.

FIG. 9 shows a schematic depicting how the integrated diagnostic platform can be adapted to a direct-to-amplification protocol. (a): Sample is mixed in dilution buffer prior to heat lysis. The heat used for sample lysis is initiated when the protocol is started in the instrument program. This and subsequent automated steps follow a specific protocol timeline that is programed into the software. (b): Following heat lysis, the lysis cartridge is inserted into the sample inlet of the diagnostic device, draining the lysate into a microchannel (paper or capillary). The lysate wicks along the microchannel into the reaction chamber. (c): The rehydration blister pouch is burst and drained into the reaction chamber, which is then heated to run the amplification reaction. The timing of reaction heating is pre-determined by the protocol program. The progression of lysate fluid and rehydration of reaction compounds is known to take a specific amount of time, which will be represented in the timing of the automated program. (d): The reaction chamber is connected to another microchannel via a magnetic valve, wicking amplicons towards the sample pad of the lateral flow assay. The magnetic valve is actuated by a magnet housed in the handheld instrument. One mode of magnetic valve actuation is to mount a magnet to a lead screw connected to a stepper motor. Being highly controllable and precise, the movements of the stepper motor-lead screw mechanism will occur at specifically programed times built into the instrument software. Alternatively, a sequence of automated electromagnets can be used in place of a magnet mounted to a stepper motor-lead screw mechanism. The running buffer blister is then burst, driving amplicons through the detection strip for the user to read and interpret results.

FIG. 10 shows a graph in which human DNA recovered from laminated borosilicate glass microspheres is quantified.

FIGS. 11A-11F show schematic illustrations of an exemplary reusable handheld controller. FIG. 11A illustrates a top view, FIG. 11B illustrates a second view, FIG. 11C illustrates an internal view of an instrument that uses a sequence of automated electromagnets rather than a stepper motor-lead screw mechanism, FIG. 11D illustrates a top view with a consumable diagnostic device, FIG. 11E illustrates a side view with a consumable diagnostic device, and FIG. 11F illustrates a third view with a consumable diagnostic device.

FIGS. 12A-12C show schematic illustrations of an exemplary a consumable diagnostic device that performs nucleic acid enrichment prior to target amplification via a laminated glass microsphere membrane mounted to a sliding stage. FIG. 12A illustrates a top view, which demonstrates the sample—in answer-out functionality with only a single test line, though multiple test lines have been used for multiplex tests. FIG. 12B illustrates a second view. FIG. 12C illustrates an exploded view of the diagnostic device. In the exploded view, it can be seen how fluid and analyte movements can be integrated using a sequence of moving parts and microchannels, leading from sample input to a lateral flow assay output that can be read through a viewing window. In this design, sponge reagent reservoirs are replaced with funnel-shaped hoppers. These hoppers operate similarly to the sponges, acting as temporary storage for assay reagents, holding wash and elution buffers until the sample processing stage is brought into fluidic connection with each hopper output, wicking buffer through the laminated silica microsphere membrane. All channels and chambers are enclosed with caps to prevent evaporation and cross-contamination.

### DETAILED DESCRIPTION

The present invention provides point-of-need diagnostic devices for detecting a target nucleic acid sequence in a sample. The devices extract, purify, elute, and amplify target nucleic acids and provide a diagnostic readout. The devices are designed to require few user steps, with each step requiring little to no effort from the operator. Advantageously, the device is hand-held.

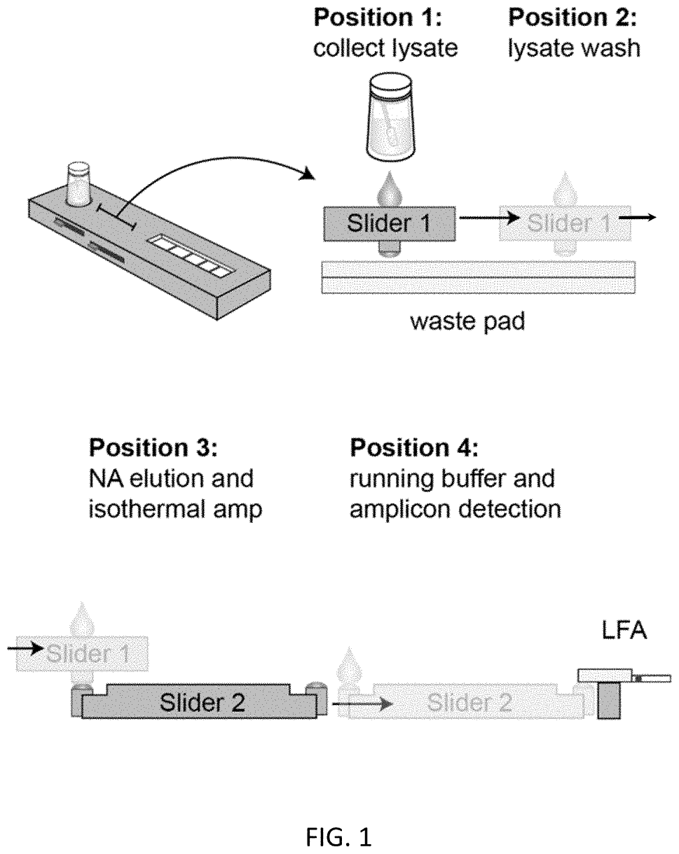

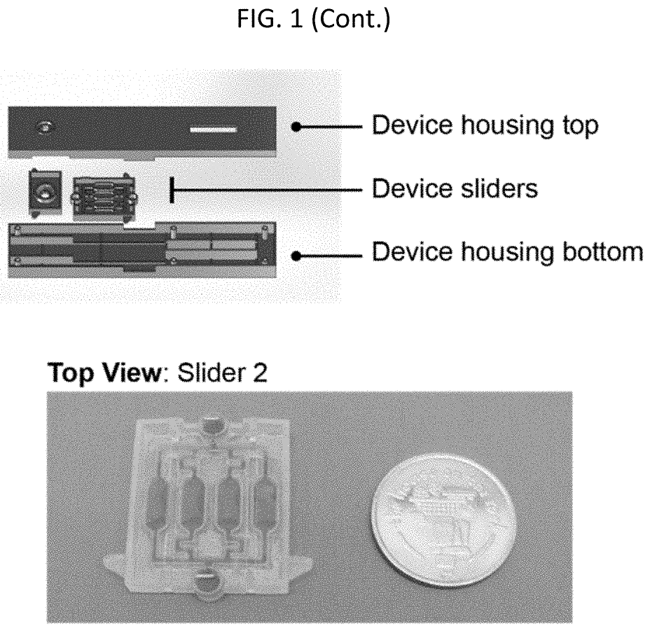

The devices comprise a housing with a sample inlet for receiving the sample. The housing contains the following within it: (a) a moveable nucleic acid binding stage comprising a permeable nucleic acid binding substrate and an eluate outlet by which an eluate may exit the binding stage; (b) a wash reservoir containing a wash buffer; (c) an elution reservoir containing an elution buffer; (d) a moveable amplification stage comprising an eluate inlet for receiving the eluate from the nucleic acid binding stage, at least one reaction chamber comprising a nucleic acid amplification reagent, and an amplicon outlet by which an amplified sample may exit the amplification stage; (e) a running buffer reservoir containing a running buffer; and (f) a detection device that provides a readout that indicates whether the target nucleic acid is present in the sample. Importantly, the nucleic acid binding stage is configured such that it can be positioned to be in fluid communication with the sample inlet, the wash reservoir, the elution reservoir, and the amplification stage via the eluate outlet. Additionally, the amplification stage is configured such that it can be positioned to be in fluid communication with the running buffer reservoir and the detection device via the amplicon outlet. See FIG. 1 for a schematic illustration of one exemplary device.

As used herein, the term “housing” refers to a rigid casing that encloses and protects the device components. The housing may comprise a sample inlet for receiving a sample or sample lysate. Advantageously, the devices of the present invention are self-contained, limiting the potential for exposure of the user to any harmful or infectious materials found in the sample. Suitably, the housing may comprise one or more readout windows that allow a user to inspect a readout of a detection device within the housing thereby avoiding the need to open the housing and potentially expose the user to harmful or infectious materials.

The nucleic acid binding stage is initially positioned directly beneath the sample inlet to receive the sample and direct it into the permeable nucleic acid binding substrate. This substrate binds nucleic acids and filters out large particulate as the sample seeps through it. Advantageously, the nucleic acid binding substrate can be positioned at the bottom of a funnel built into the nucleic acid binding stage, such that the sample is directed into the substrate. The nucleic acid binding substrate may comprise any permeable material that can bind nucleic acids in a complex matrix and can release the nucleic acids when contacted with an eluent. Advantageously, the substrate binds nucleic acids in a nonspecific manner, allowing it to be used to separate any nucleic acid from a sample without having to be tailored for a specific nucleic acid target. Suitable nucleic acid binding substrates include, without limitation, cellulose membranes, cellulose-coated beads, chitosan membranes, chitosan-coated beads, silica columns, aluminum oxide membranes, and the like. In some embodiments, the nucleic acid binding substrate is a silica membrane and/or silica microspheres. For example, borosilicate glass microspheres may be used for DNA extraction as disclosed in International Patent Pub. No. WO/2019/109092, which is incorporated by reference herein.

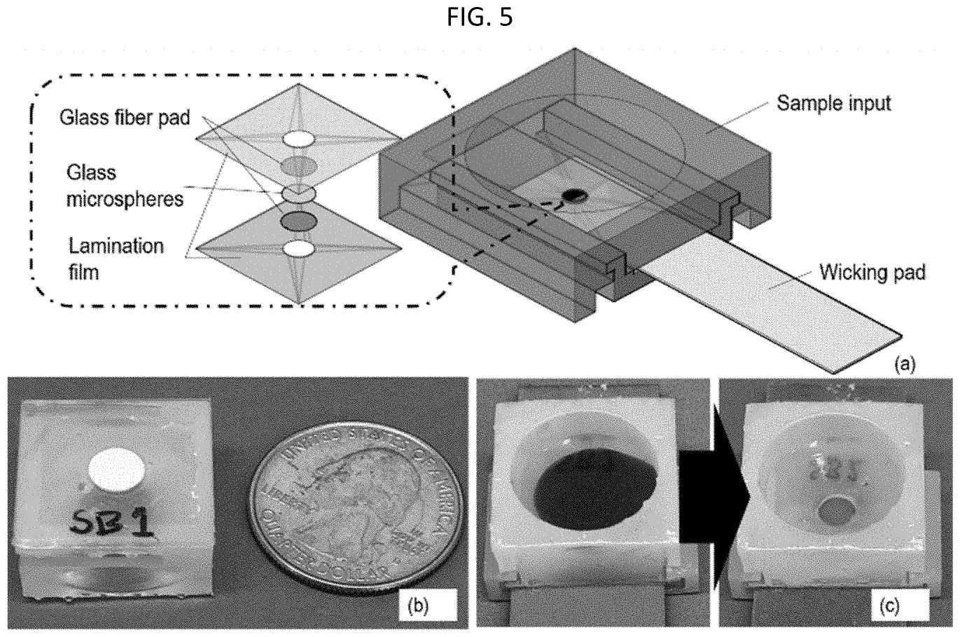

In some embodiments, the binding substrate comprises silica microspheres and fiber pads (e.g., paper, silica, cellulose, etc.) or membranes. This substrate combines the advantages of silica-bead-based and paper-based nucleic acid extractions while eliminating their drawbacks. To form this substrate, micrometer-sized silica beads are packed between two pieces of fiber pads via a low-cost, readily deployable lamination process (FIG. 5). The laminated beads will bind nucleic acids and allow the use of a paper, or a similar material, with larger pore sizes for fast flow and clogging prevention. The use of fiber pads and lamination not only simplifies the silica beads packing process but also enables the use of capillary forces for liquid transfer. The binding of nucleic acids is achieved using a combination of chaotropic agents, such as guanidium isothiocyanate and ethanol, and a co-precipitant such as glycogen and Glycoblue™. Exemplary nucleic-acid-capture microspheres and their production and use are described in International Patent Pub. No. WO/2019/109092.

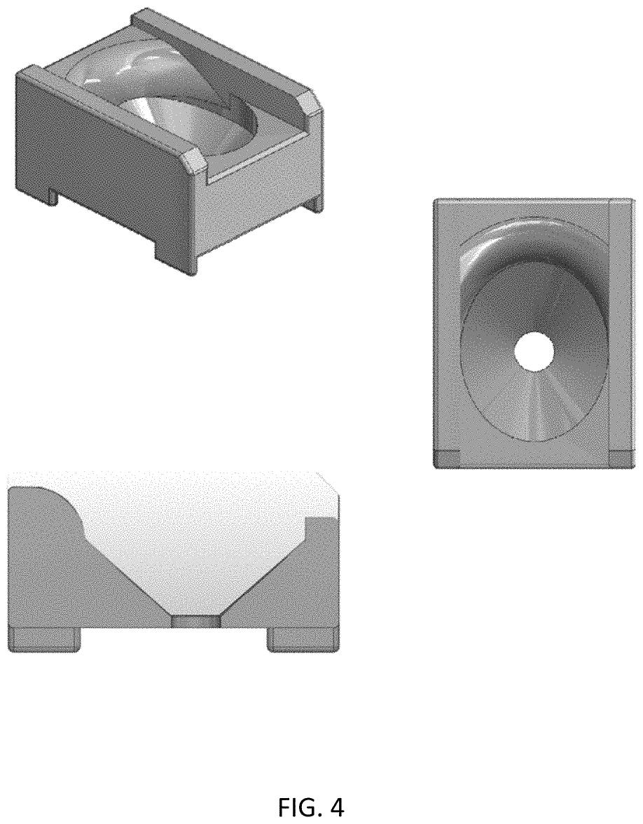

The binding stage may comprise a collection funnel, binding membrane, and sponge ramp (FIG. 4). The funnel collects liquids as they are introduced to the binding stage. First in the liquid collection sequence, the lysate drains from the lysis cartridge, through the inlet of the processing/detection device and into the collection funnel. The funnel channels the lysate through the binding substrate, which collects nucleic acids as the lysate passes through. The devices are designed to be operated by simple, limited, manual interventions, such as sliding a movable component or pressing down a collapsible chamber. In some embodiments, the device may comprise two moveable stages: the nucleic acid binding stage and the amplification stage. In some embodiments, the nucleic acid binding stage is configured to slide between positions of fluid communication with the sample inlet, the wash reservoir, and the elution reservoir, and the amplification stage is configured to slide between positions of fluid communication with the elution reservoir, the running buffer reservoir, and the detection device.

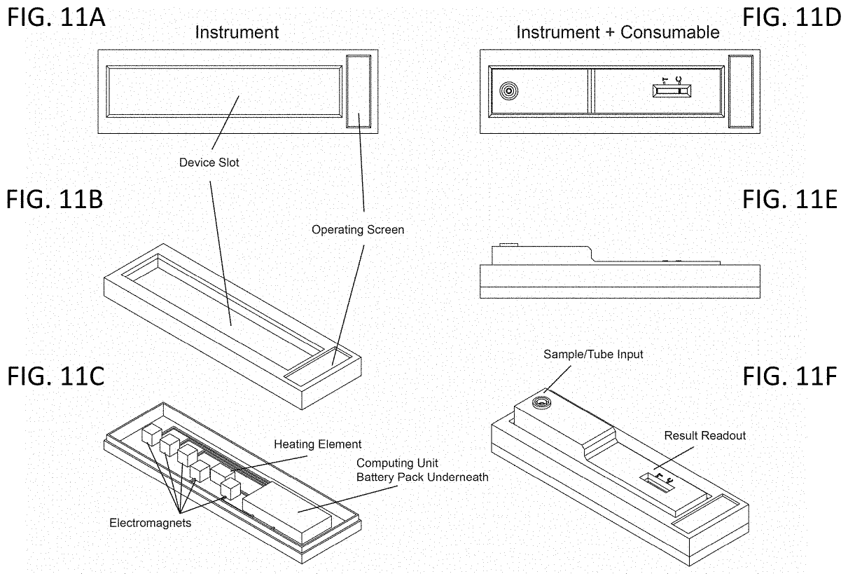

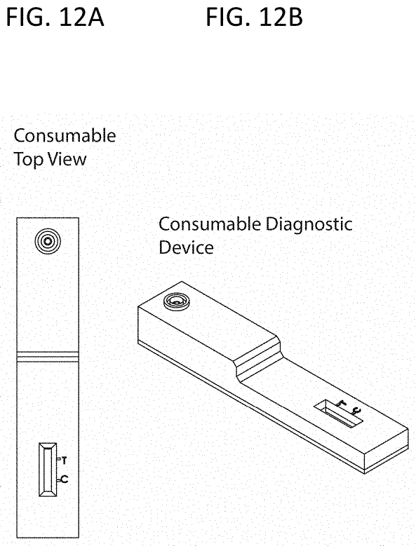

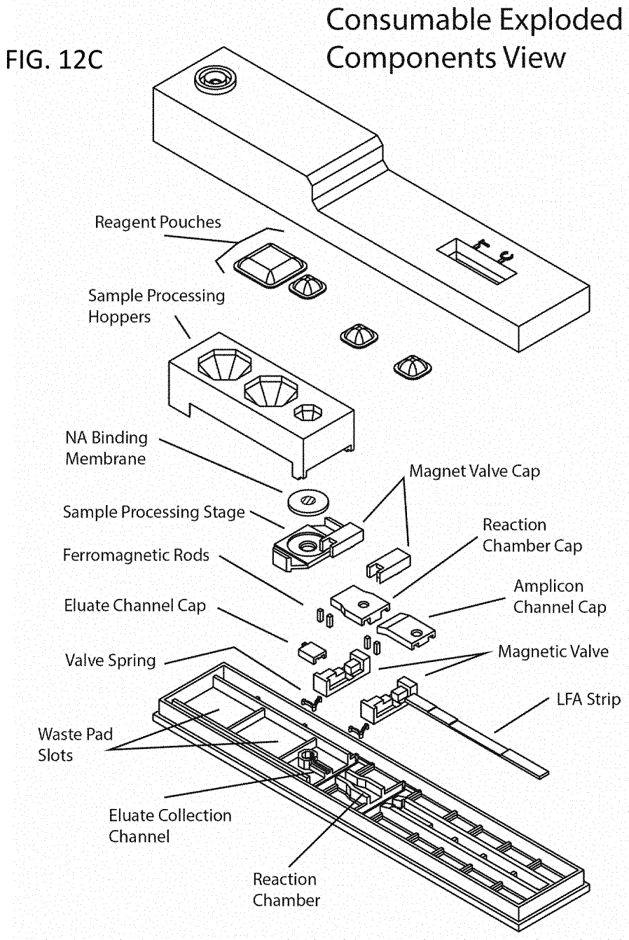

In some embodiments, the device is intended to be used with a reusable, handheld controller (FIG. 2, FIG. 11) that moves internal components within the disposable processing/detection device (FIG. 12) in an automated fashion. The reusable controller removes user manual steps previously required, simplifying platform operation, which increases success rate by removing variability. The handheld platform is portable so it can be used throughout a single clinic or in mobile field clinics, among other unique diagnostic settings.

The binding stage or amplification stage may be controlled using a handheld controller and moved using a force, such as mechanical or electromagnetic force. Stage movements may be automated by a single-board microcontroller that is coded to control a small electric motor, such as a stepper motor. The small electric motor may spin a lead screw that is threaded with a moving stage. Features attached to this moving stage, such as a magnet or clips or similar physical features, connect with the binding and amplification stage within the processing/detection device. The program installed onto the microcontroller moves the electric motor at specific speeds at specific times, according to the protocol designed to collect, amplify and detect target nucleic acid sequences.

The microcontroller, electric motor, and external force application stage are all housed within a reusable device or controller. The controller will be made of polymer material that can be easily decontaminated with standard wipes or spray, preventing cross-contamination and the risk of passing infections from one person to another. The reusable controller is designed to be portable so one can be used throughout a doctor's office or in a mobile clinic setting. The reusable controller has a user interface and buttons that allows the user to select a protocol specific to a sample or target type.

The heating element that is required for nucleic acid amplification will be powered by the reusable controller. The battery pack that powers the microcontroller and the electric motor also powers the heating element. The microcontroller will command the temperature and reaction time. The heating element could be housed within the controller or within the processing/detection device and connected to the power source via external nodes.

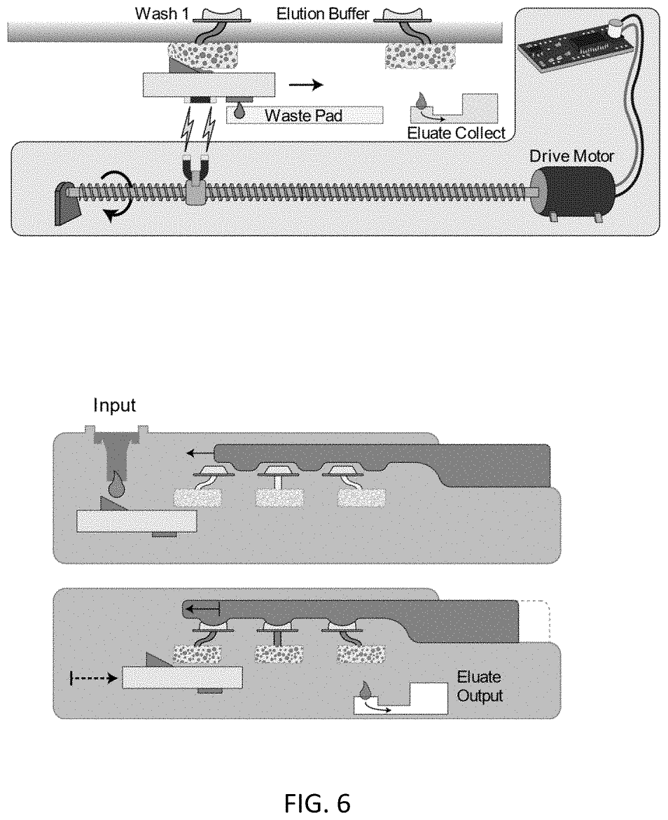

Additionally, the device may comprise one or more reservoirs. In some embodiments, the device comprises four buffer reservoirs: the wash reservoir, elution reservoir, rehydration buffer reservoir, and running buffer reservoir. A reservoir is suitably liquid-tight and/or airtight to allow the contents to be maintained for extended periods of time in ambient conditions until needed for use. In some embodiments, one of more of these reservoirs comprise a blister pouch (e.g., an aluminum foil blister pouch). Suitably the reservoir's contents can be accessed when the appropriate stage is positioned to be in fluid communication with the reservoir. For example, the stage may be configured to puncture the blister pouch when the stage is in position to receive the reservoir's contents. Prepackaged buffers can be strategically incorporated along the sliding path of each stage. In this configuration, the operator slides the stages against the buffers reservoir, puncturing the pouches to deliver the proper volume of each buffer in sequence (FIG. 1). In another embodiment, reservoirs are pierced and drained into sponges for temporary storage as they await their turn in the protocol sequence. The sponge ramp gently compresses the sponges, draining reagents into the funnel of the binding stage (FIG. 6). The sponge ramp of the binding stage may be built up from the collection funnel. The purpose of the ramp is to compress reagent sponges as the binding stage moves along the protocol track. The reagent sponges are a passive way to temporarily store reagents after they are expelled from their shelf-life storage reservoirs. To optimize the work done by the limited number of user steps required for a CLIA-waived point-of-need device, some or all of the shelf-life reagent reservoirs are burst all at once. The reservoirs are pierced and drained into the reagent sponges through mechanical actuation as the processing/detection device is inserted into a reusable controller. From there, the sponges saturate with their respective reagent, holding it until compressed by the sponge ramp. As the ramp moves beneath a sponge that is saturated with a specific buffer, it gently compresses the sponge, draining it of a set volume of liquid into the stage. The drained reagents flow into the collection funnel via gravity and are wicked through the binding membrane via fluidic forces.

The wash reservoir may contain any suitable wash buffer for nucleic acid extraction. As used herein, a “wash buffer” is a substance capable of removing impurities adsorbed onto the surface of the nucleic acid binding substrate. The wash buffer should be selected such that nucleic acids adsorbed onto the nucleic acid binding substrate are not extracted when the wash buffer contacts the substrate. Suitably, the wash buffer may be selected from water, an alcohol (e.g., ethanol or isopropanol), medium salt buffer (e.g., 100 mM or 200 mM NaCl), or combinations thereof. In some embodiments, the wash buffer is ethanol diluted to a concentration of 30% or less by volume.

The elution reservoir may contain any suitable elution buffer that is able to separate nucleic acid material from the nucleic acid binding substrate. The elution solution is preferably an aqueous solution of low ionic strength. In some embodiments, the elution buffer is water or TE buffer (i.e., Tris-HCl ethylenediamine-tetraacetic acid (EDTA)). However, other elution buffers suitable for use with this invention will be readily apparent to one skilled in this art.

The running buffer reservoir may contain any running buffer that is compatible with the detection device. Suitable running buffers include, without limitation, water, phosphate buffered saline (PBS), saline-sodium citrate (SSC) buffer, Tris buffer, MES buffer, HEPES buffer, or any other buffer suitable for buffing at a pH between 7.4 and 8.0. In some embodiments, the running buffer is a buffered salt solution containing a detergent, such as 75 mM Sodium Borate, pH 7.4, 0.25% Tween 20.

In some embodiments, the device includes a waste pad positioned beneath the nucleic acid binding stage to absorb liquid (i.e., sample and buffer) that passes through the nucleic acid binding substrate. The waste pad may take any form and may comprise any absorptive material, provided that it does not disrupt device function.

The device comprises at least one reaction chamber in which a nucleic acid amplification reaction may be performed. The reaction chamber contains a nucleic acid amplification reagent. In some embodiments, this reagent comprises the basic factors required for DNA amplification, i.e., a DNA polymerase, one or more primers, and nucleoside triphosphates. In some embodiments, the nucleic acid amplification reagent is lyophilized and is reconstituted as the eluate flows into the reaction chamber. Use of a lyophilized reagent is advantageous for a point-of-need device because such reagents remain stable for a long time at room temperature.

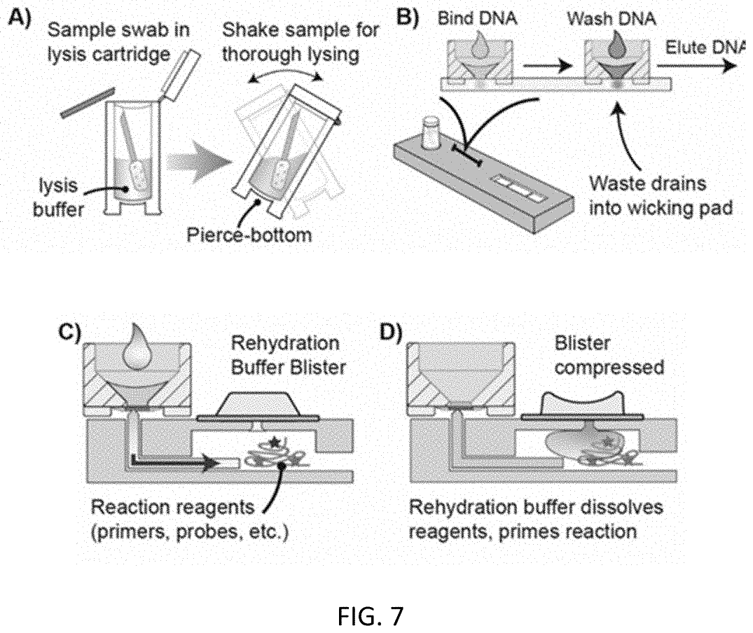

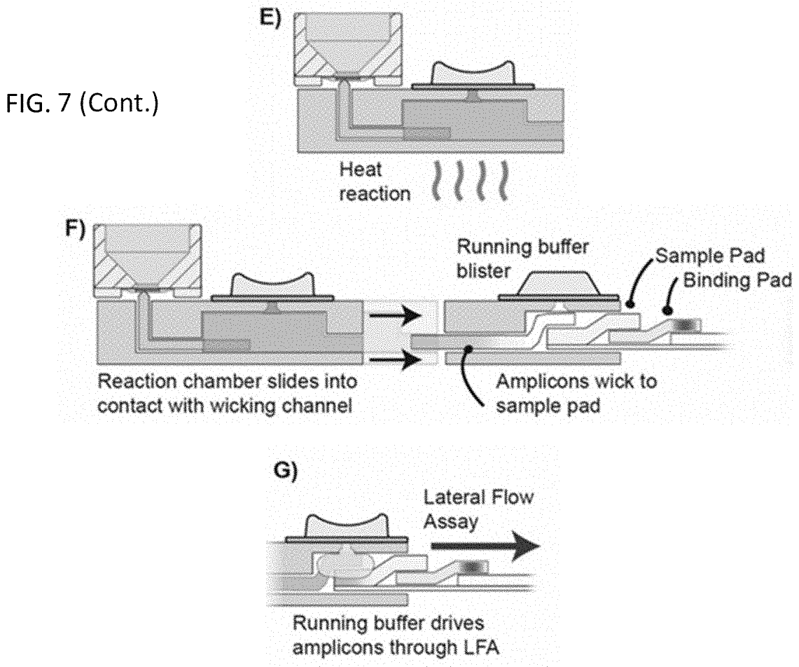

After the extracted nucleic acids have been washed, the outlet of the binding stage is positioned above the inlet of the reaction stage. When this occurs, a fluidic connection is made between the two stages, draining eluate from the binding substrate into a capillary microchannel. The microchannel wicks eluate via capillary action into one or several reaction chambers. This wicking could be done through any sort of hydrophilic microchannel. One such example is a paper microchannel that comes into contact with the bottom of the binding membrane (FIG. 7)

The reaction chambers may be pre-packaged with lyophilized reaction reagents—including primers, probes, and magnesium. The rehydration buffer reservoir may be pierced, draining into the reaction chamber and mixing all reagents. Once the rehydration reservoir is burst, the heating element begins warming the reaction to the desired temperature for the desired duration. The reaction temperature and duration are specific for the desired nucleic acid target that is being amplified.

Once the reaction is complete, the reaction chamber is brought into fluidic contact with a capillary microchannel, similar to the previous microchannel (FIG. 7). This downstream microchannel wicks the reaction products (i.e., amplicons) towards the sample pad of the lateral flow assay used for target detection. After the amplicons have been wicked into the sample pad, the running buffer reservoir is pierced. As the running buffer drains into the sample pad it drives the amplicons to wick through the binding pad and eventually through the test strip of the lateral flow assay. The lateral flow assay then works as any standard lateral flow diagnostic test. The user can read the test lines that arise over the next few minutes and determine results. The device could also be read by an automated reading device that is built into the reusable controller.

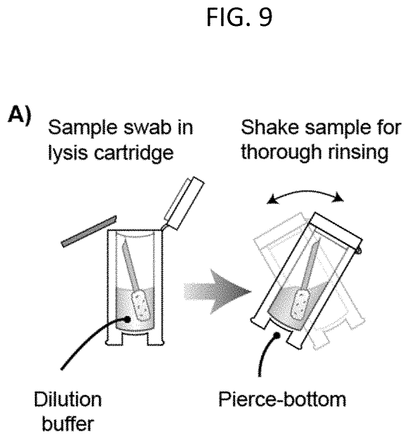

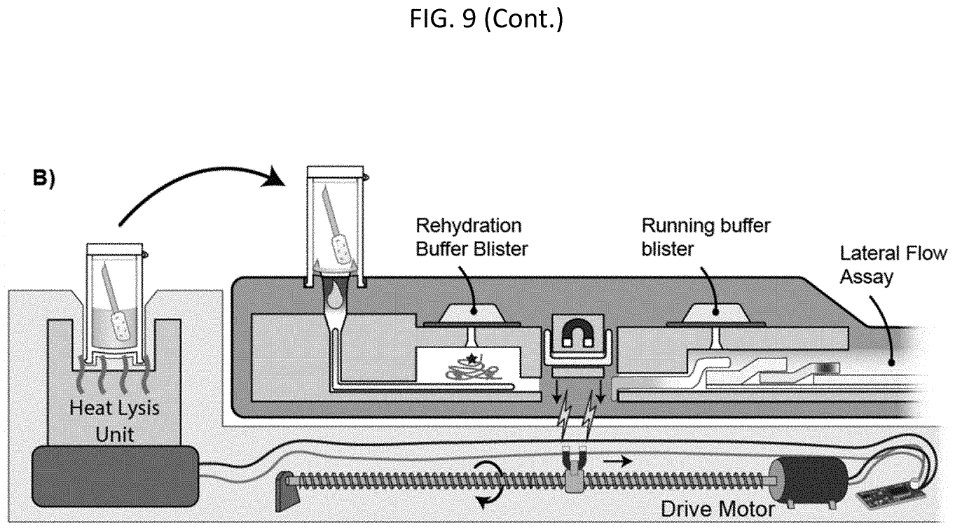

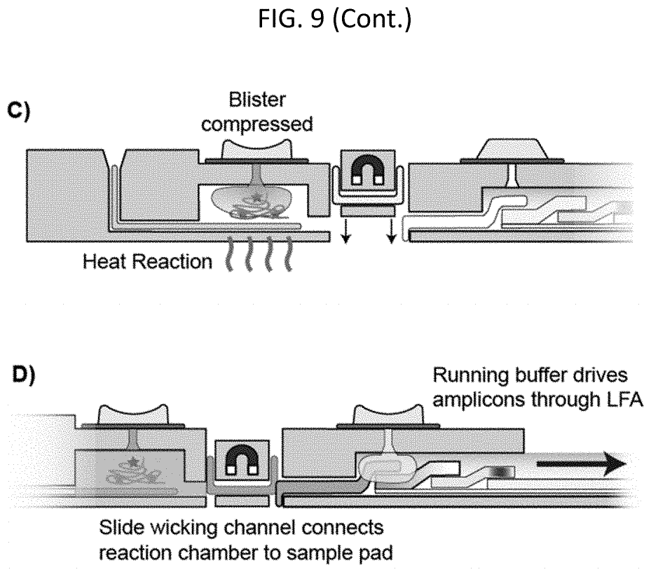

Another exemplary embodiment of the invention is illustrated in FIG. 9. This embodiment takes advantage of “direct-to-amplification” assay techniques and does not require nucleic acid extraction, wash, or elution, thus, reducing the number of automated steps. This platform is best suited for samples that are more conducive to direct-to-amplification techniques, such as nasal swabs, nasopharyngeal swabs, oropharyngeal swabs and saliva (FIG. 9A). Here, the sample will be heat lysed within the lysis cartridge in a heating slot built into the reusable controller (FIG. 9B). Following lysis, the tube is transferred to the sample inlet of the diagnostic device. Note how, in this design, the sample inlet is in direct fluidic connection with the reaction chamber. This is because no nucleic acid extraction, washing, or elution is needed prior to amplification. As before, the cartridge is pierced upon insertion, draining an aliquot of sample into the reaction chamber.

Once enough lysate has wicked into the reaction chamber, the rehydration buffer blister pouch is burst and drained, filling the chamber and mixing the reagents (FIG. 9C). The reaction is then heated to the required temperature for efficient amplification to occur. Following amplification, the reaction chamber is put into fluidic connection with the lateral flow assay via another wicking channel (FIG. 9D). Once amplicons have reached the sample pad, the running buffer blister is burst, driving amplicons along the lateral flow assay, through the detection strip. The strip results are then read by the user or an automated strip reader. Medical action may be taken based on the strip results.

Any suitable DNA polymerase may be included in the nucleic acid amplification reagent. As used herein, “DNA polymerase” refers to an enzyme capable of catalyzing the formation of DNA. In embodiments in which the amplification step is performed under isothermal conditions, the DNA polymerase is advantageously a strand-displacing polymerase (i.e. a polymerase with the ability to displace downstream DNA encountered during synthesis). Exemplary strand-displacing DNA polymerases include phi29, Bst, Bsm, Bsu, and Klenow fragment.

As used herein, a “primer” means a nucleic acid designed to bind via complementarity to sequences that flank the target sequence in the template nucleic acid. During amplification, polymerases extend primers. The primer's binding site should be unique to the target sequence with minimal homology to other sequences to ensure specific amplification of the intended target sequence. In some embodiments, the nucleic acid amplification reagent may comprise at least one primer that is detectably labeled. Exemplary labeling moieties include, without limitation, a gold nanoparticle, a protein binding ligand, a hapten, an antigen, a fluorescent compound, a dye, a radioactive isotope, and an enzyme. In some embodiments, the nucleic acids are amplified using a labeled primer set (comprising a forward and a reverse primer), generating amplification products with tagged primers at both ends for easy detection via a lateral flow device. Primers may further comprise nuclease cleavage sites and/or blockers (e.g., phosphoramidite blocker) to provide amplification specificity.

In embodiments in which the target nucleic acid is RNA, the eluate may be subjected to a reverse transcription reaction to generate complementary DNA (cDNA) prior to amplification (i.e., reverse transcription polymerase chain reaction). In these embodiments, the nucleic acid amplification reagent may comprise a reverse transcriptase and at least one suitable primer. A “reverse transcriptase” is an RNA-dependent DNA polymerase. The reverse transcriptase initiates synthesis of a DNA transcript using the RNA as a template, forming a single-stranded cDNA. A double-stranded DNA molecule may be produced from the cDNA using a DNA polymerase. Either the single-stranded cDNA or the double-stranded DNA prepared from reverse transcription may serve as the input for the subsequent amplification reaction.

Standard methods of nucleic acid amplification require (1) high temperatures to increase reaction kinetics and expedite primer-target annealing and (2) expensive laboratory equipment, such as heating blocks, centrifuges, bead-beaters, magnetic beads, and/or volume dispensing robots. To be more suitable for use in a point-of-need device, an amplification method must be free of such requirements. To this end, the devices of the present invention may be configured such that the nucleic acid amplification step is performed isothermally, without the use of a thermocycler. As used herein, “isothermally” or “under isothermal conditions” means that reaction is conducted at a relatively constant temperature. Suitably, the reaction is conducted with temperature fluctuations less than ±10° C., ±5° C., or ±2° C. In some embodiments, the amplification methods are performed without any equipment requiring a power supply to provide source heat for the amplification reaction. In some embodiments, the methods are performed at a temperature below 70° C., 65° C., 60° C., 55° C., 50° C., 65° C., or 40° C. In certain embodiments, the methods are performed at a temperature below 37° C. Suitably, the methods may be performed at a temperature between 20° C. and 70° C., 20° C. and 65° C., 20° C. and 60° C., 20° C. and 55° C., 20° C. and 50° C., 20° C. and 45° C., 20° C. and 40° C., or 20° C. and 37° C.

Any isothermal amplification technique may be used with the present invention. Suitable known techniques include loop-mediated isothermal amplification (LAMP), reverse-transcriptase loop-mediated isothermal amplification (RT-LAMP), recombinase polymerase amplification (RPA), strand displacement amplification (SDA), helicase-dependent amplification (HDA), nucleic acid sequence based amplification (NASBA), nicking enzyme amplification reaction (NEAR), and transcription-mediated amplification (TMA). Details of such isothermal amplification techniques can be found in Zhao et al. Chem. Rev. 2015, 115, 12491-12545 and Craw and Balachandran Lab Chip, 2012, 12, 2469-2486. Alternatively, isothermal amplification may be performed using the annealase-mediated methods disclosed in U.S. Provisional Patent Application 62/895,778, incorporated by reference herein.

With any isothermal amplification technique, the annealase ICP8 and, optionally, a helicase may be included to accelerate the reaction. ICP8 is derived from the herpesvirus DNA replication system. This annealase promotes efficient replication of the viral genome during host cell infection by stabilizing single-stranded DNA (ssDNA) and recruiting various factors necessary for replication. Specifically, ICP8 binds ssDNA, samples ssDNA for base pairing, and anneals to ssDNA molecules. Thus, ICP8 can be used to promote the annealing of DNA primers to their complementary targets during an amplification reaction. By increasing reaction kinetics and reducing off-target amplification, ICP8 allows the reaction to be performed at a lower temperature with increased specificity. The ICP8 used with the present invention may be from any available source, including from any herpesvirus or another closely related virus. For instance, the ICP8 may be derived from chelonid herpesvirus 5, a type of herpesvirus that infects the Hawaiian green sea turtle, which has an internal body temperature of 20-25° C.

Since ICP8 lacks helicase function, helicases or nucleases must be added to the reaction to generate ssDNA for ICP8 to sample. Any suitable helicase or nuclease may be used with the methods of the present invention. Exemplary helicases include, without limitation, UvrD, RecBCD, BLM, WRN, and RecQ. Exemplary nucleases include UL12, nickases, and restriction enzymes.

The largest limitation to the selection of enzymes for use in the amplification reaction will be their ability to function at the temperature at which the devices are intended to be used. However, it is standard practice for one of skill in the art to optimize reaction conditions and enzyme components to achieve particular reaction goals (i.e., sensitivity, specificity, speed, and efficiency at a given temperature). For instance, conditions such as primer length, melting temperature (Tm), GC content, reaction buffering conditions (e.g., pH, salt concentrations, dNTP concentrations), and the presence of crowding agents (PEG) can be varied. Literature regarding isothermal amplification reactions and ICP8 DNA binding, strand-invasion, and recombination assays may provide guidelines that aid in reaction optimization.

The nucleic acid amplification reagent may further comprise additional components, including cofactors, buffering agents, amplification enhancers, or any combination thereof. As used herein, a “cofactor” is a substance other than the substrate that is essential for the activity of an enzyme. Suitably, the cofactor may be magnesium, which functions as a cofactor for a variety of polymerases. The cofactor may be introduced to the amplification reaction as a salt, e.g., MgSO₄ or MgCl₂. As used herein, a “buffering agent” comprises a weak acid or base used to maintain the acidity (pH) of a solution near a chosen value after the addition of another acid or base. Suitably, the buffering agent may be selected from Tris-HCl, (NH₄)₂SO₄, or KCl. As used herein, an “amplification enhancer” is a substance that may enhance amplification specificity, efficiency, consistency, and/or yield. Suitably, the amplification enhancer comprises dimethyl sulfoxide, glycerol, formamide, polyethylene glycol, N,N,N-trimethylglycine (betaine), bovine serum albumin, tetramethylammonium chloride, a detergent, or combinations thereof. Suitably, the detergent is a nonionic detergent such as Tween 20 or Triton X-100.

Any detection device that provides a readout that indicates whether a target nucleic acid is present in a sample may be used with the present invention. The presence of the target nucleic acid may be detected by any suitable method or assay technique, including, without limitation, a binding assay, a colorimetric assay, an electrophoretic assay, a fluorescence assay, a turbidity assay, an electrochemical assay, and the like. Detection devices may provide an analog or digital readout.

In some embodiments, the detection device is a lateral flow device. As used herein, a “lateral flow device” is a porous device capable of detecting the presence of a target nucleic acid sequence traversing a series of beds. Lateral flow devices typically comprise (a) a sample loading area at one end; (b) an area comprising a detectably labelled probe specific for a target nucleic acid sequence, wherein said detectably labelled probe is not bound to the lateral flow device and is capable of wicking across the lateral flow device; (c) an area comprising a capture probe for the target nucleic acid sequence, wherein said capture probe for the target nucleic acid sequence is immobilized on the lateral flow device; and (d) absorbent material, wherein the absorbent material wicks an aqueous sample across the lateral flow device when the aqueous sample is added to the sample loading area. Thus, in some embodiments, the lateral flow device comprises a sample loading area, an amplification area, a solid support, an absorbent sample pad, or any combination thereof. A detailed description of exemplary lateral flow devices can be found in U.S. Patent Publication No. 2018/0148774, incorporated by reference herein.

In embodiments that utilize a lateral flow device, the test results may be displayed using lateral flow assay (LFA) strips, which provide a readout similar to that of a pregnancy test strip. The strips comprise a capture probe for the target nucleic acid sequence, wherein said capture probe is immobilized on the lateral flow device in a region referred to as the “test area”. The test area can be in any form with well-defined boundaries, such as a dot or a strip. The capture probe may be immobilized on the lateral flow device by covalent coupling or affinity binding. Suitably, the capture probe is attached to the lateral flow device by biotin:streptavidin affinity binding. Generally, the capture probe is capable of specifically hybridizing to part of the target DNA sequence, separate from the detector probe sequence to which the detectably labelled primer will bind. The LFA strip may comprise multiple probe-capture lines designed to capture different target sequences.

In some embodiments, the amplified RNA or DNA are dual-labeled using two primers that have a biotin label on one primer and a second label (e.g., a FITC, DIGO or TAMRA tag) on the other reverse primer. The streptavidin conjugated AuNPs/latex bead (the colorimetric moiety) will bind to the biotin side of the amplicons while the tag molecule on the other side is captured by an antibody (anti-FITC or anti-DIGO or anti-TAMRA, respectively) attached to the strips. The rest of the streptavidin conjugated AuNPs/latex bead will be captured by the biotin control line on the LFA.

In some embodiments, the detection device is configured such that detection is accomplished by visual inspection, either with or without additional instrumentation. For example, results can be quantified by imaging and analysis with a computer. In some embodiments, the result can be scanned with a smartphone and electronically sent to a clinician, for example, with a computer that has an Adobe Acrobat grayscale converter or an Image J image processing software to quantify the visible light signal from a gold nanoparticle. Likewise, a color wheel for visualization of positive tests may be utilized.

In some embodiments, the devices further comprise a heating element. For use in a point-of-need device, the heating element is advantageously portable and does not require electricity. In particular embodiments, the heating element comprises a battery-powered heating film. In alternative embodiments, the heating element may use a reversible or irreversible exothermic chemical reaction to generate heat.

### Kits:

The present invention also provides kits comprising the point-of-need diagnostic devices disclosed herein and further comprising one or more of a lysis cartridge, a lysis agent, a collection device, a heating element, and a handheld controller. These optional, additional components may be included to ensure that use of the kit is safe, simple, and hands-off. This is of particular importance when the samples used with the present invention may contain harmful or infectious materials.

In some embodiments, the kits include a collection device. The collection device may comprise any suitable device for containing the sample, such as a container, specimen jar, syringe, needle, bag, specimen collection paper, or swab. In some embodiments, the collection device comprises a Puritan® HydraFlock swab, which is designed for absorption and retention of cellular material. Since this swab can hold approximately 250 μL of sample when fully saturated, its use standardizes sample input without requiring any measurement or transfer of infectious liquids.

In some embodiments, the kits include a lysis cartridge comprising a lysis agent. Advantageously, the lysis cartridge provides a simple, hands-off means to lyse cellular components within the sample. The lysis cartridge may be configured to allow for direct insertion of a sample (e.g., via a swab or needle). To promote lysis, the operator may be instructed to cap and shake the cartridge. As used herein, a “lysis agent” is a composition capable of breaking down or disrupting a cellular membrane. Ideally, the lysis agent results in efficient cell lysis without the use of any equipment, such as a heating block or vortex. Suitable lysis agents include, without limitation, chaotropic salts (e.g., guanidine thiocyanate, alkali metal perchlorates, alkali metal iodides, alkali metal trifluoroacetates, alkali metal trichloroacetates, alkali metal thiocyanates, urea, guanidine HCl, guanidine thiocyanate, guanidium thiosulfate, and thiourea), lytic enzymes (e.g., beta glucurondiase, glucanase, glusulase, lysozyme, lyticase, mannanase, mutanolysin, zymolase, cellulase, lysostaphin, pectolyase, and streptolysin O), and detergents (e.g., sodium dodecyl sulfate (SDS), NP-40, 3-[(3-cholamidopropyl)dimethylammonio]-1-propanesulfonate, octyl-β-thioglucopyranoside, octyl-glucopyranoside, 3-(4-heptyl) phenyl 3-hydroxy propyl) dimethylammonio propane sulfonate, 3-[N,N-dimethyl(3-myristoylaminopropyl)ammonio] propanesulfonate, 3-(decyldimethylammonio)propanesulfonate inner salt, 3-(dodecyldimethylammonio)propanesulfonate inner salt, 3-(N,N-dimethylmyristylammonio)propanesulfonate, and n-dodecyl a-D-maltoside). In some embodiments, the lysis agent is a chaotropic salt.

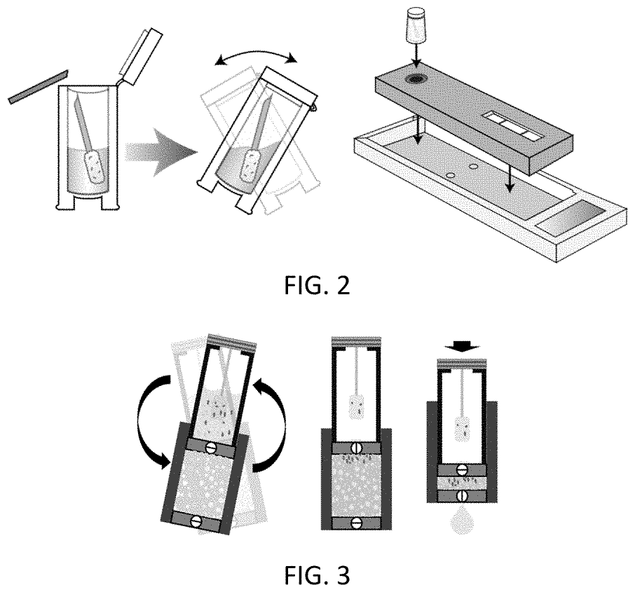

In some embodiments, the lysis cartridge can be used with samples that require particulate filtration, such as stool (FIG. 3). The lysis cartridge may make use of a small pore filter, such as a filter membrane or a porous foam filter. When lysate is drained from the lysis cartridge, it is forced through the particulate filter through a plunging action. This plunging action resembles how a syringe is evacuated by creating positive pressure within the cartridge chamber, forcing lysate through the filter and into the inlet of the processing/detection device. The filter can also be embedded with resin, activated charcoal or something similar for the removal of non-particulate, chemical contaminants. Complex samples, such as stool, contain reaction inhibitors like bile salts. The removal of such inhibitors can greatly improve nucleic acid recovery and amplification reaction efficiency.

### Methods:

Methods of using the point-of-need diagnostic devices or the kits disclosed herein to detect a target nucleic acid sequence in a sample are also provided with the present invention. The methods involve (a) loading a sample lysate into the diagnostic device via the sample inlet, thereby contacting the sample with nucleic acid binding substrate; (b) positioning the nucleic acid binding stage to be in fluid communication with the wash reservoir, thereby contacting the nucleic acid binding substrate with the wash buffer; (c) positioning the nucleic acid binding stage to be in fluid communication with the elution reservoir and the amplification stage, thereby contacting the nucleic acid binding substrate with the elution buffer and eluting the eluate; (d) heating the eluate and the nucleic acid amplification reagent in the reaction chamber under conditions sufficient for amplifying the target nucleic acid, thereby preparing the amplified sample; (e) positioning the amplification stage to be in fluid communication with both the running buffer reservoir and the detection device, thereby transferring the amplified sample to the diagnostic device; and (f) inspecting the readout provided by the detection device to determine whether the target nucleic acid is present in the sample.

The devices, kits, and methods of the present invention can be used with or without lysing the cells within a sample. However, with particular sample types and/or target nucleic acid sequences, lysis may be necessary to ensure that a sufficient amount of nucleic acid is extracted from the sample. Thus, in some embodiments, the methods further comprise contacting the sample with the lysis agent in the lysis cartridge, thereby producing a sample lysate that is loaded into the diagnostic device. In some embodiments, the loading step comprises contacting the lysis cartridge with the sample inlet to form a fluid connection between the lysis cartridge and the diagnostic device. In these embodiments, the bottom of the cartridge may be designed so that it is punctured upon contact with the sample inlet, allowing the lysate solution to flow from the cartridge down into the device.

One objective of the present invention is to provide rapid test results without the need for expensive laboratory equipment (e.g., a thermocycler). Thus, in some embodiments, the eluate and the nucleic acid amplification reagent are heated for no longer than 30 minutes and/or are heated to a temperature between 25° C. and 65° C. during the amplification reaction (step d). Further, in some embodiments, the entire method is completed in 60 minutes or less, 30 minutes or less, or 20 minutes or less. This speed could, for instance, allow a care provider to determine whether a patient is infected with a pathogen and prescribe a treatment within in a single visit.

As used herein, a “sample” is a substance that comprises or may comprise nucleic acids. The samples used with the present invention may be liquid, solid, and semi-solid samples. In some embodiments, the biological sample comprises cells in culture. In other embodiments, the sample is a biological sample obtained from a subject (e.g., a patient). Exemplary subject samples include stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool, stool water, pancreatic juice, lavage fluids from sinus cavities, bronchopulmonary aspirates, blastocyl cavity fluid, umbilical cord blood, a skin swab sample, a throat swab sample, a genital swab sample, and an anal swab sample. In some embodiments, the sample is an environmental sample from a source other than a subject. Suitably, the environmental sample may be a water sample such as from a drinking or cooking water source. In other embodiments, the environmental sample may be a food sample or other consumable sample. In other embodiments, the environmental sample is a surface sample such as may be obtained from swabbing a surface.

As used herein, a “target nucleic acid sequence” is a nucleic acid sequence indicative of an origin or source. In some embodiments, the target sequence is indicative of the presence of a particular organism, such as a pathogen (e.g., a bacterium, a fungus, a virus, or a protist). In other embodiments, the target sequence is indicative of the presence or absence of a disease or condition, such as the presence or absence of a genetic mutation associated with the disease or condition. In yet other embodiments, the target sequence is indicative of the prognosis, progression, or response to treatment for a disease or condition, such as the presence or absence of a genetic mutation or genetic marker associated with the prognosis, progression, or response to treatment for a disease or condition. As used herein, “indicative” or “indicates” means to point to or be a sign of an origin or source, whether alone or in combination with additional target sequences or other information. The target nucleic acid sequence may be found anywhere in the genome of a specific organism or virus, but it should be specific to said organism or virus. Methods for choosing a target nucleic acid sequence and designing primers to amplify a target nucleic acid sequence are common practice in the art. Many resources, including literature publications and NCBI databases such as “BLAST”, may be used to guide primer design.

The target nucleic acid sequence used with the present invention may be derived from genomic DNA (e.g., DNA encoding a protein, open reading frames, or regulatory sequences), mitochondrial DNA, extracellular DNA, plasmid DNA, or cell-free fetal DNA. Alternatively, the target nucleic acid sequence may be derived from an RNA, such as a messenger RNA (mRNA), transfer RNA (tRNA), ribosomal RNA (rRNA) or small nuclear RNA (snRNA).

The devices of the present invention may be configured to detect the presence of a multiplicity of target nucleic acid sequences. In some embodiments, the devices are configured to detect at least two, at least three, at least four, at least five, at least six, at least seven, at least eight, at least nine, at least ten, at least eleven, at least twelve, at least thirteen, at least fourteen, at least fifteen, at least twenty, or at least twenty five target nucleic acid sequences. To this end, the devices may comprise a multiplicity of reaction chambers, such that each target nucleic acid sequence can be amplified in a separate chamber. This configuration avoids primer competition and simplifies primer design. In some embodiments, at least one reaction chamber is dedicated to the amplification of a control gene (e.g., a constitutively expressed housekeeping gene). Further, the device may comprise a multiplicity of detection devices to facilitate multiplex detection.

### EXAMPLES

Human RNA isolation using laminated borosilicate glass microspheres. The RNA recovery rates from two RNA binding materials, i.e., laminated glass fiber and laminated borosilicate glass microspheres, were compared. The laminated borosilicate glass microspheres were prepared using two fabrication techniques. The first fabrication technique (the “pipette bead” method) involves pipetting borosilicate glass microspheres dispersed in water onto a glass fiber pad prior to the lamination. The second fabrication technique (the “PVA bead” method) involves gluing a borosilicate glass microsphere biscuit between two pieces of glass fiber pads together using a poly(vinyl alcohol) (PVA) solution to form a sandwich prior the lamination. The borosilicate glass microsphere biscuit is formed by mixing borosilicate glass microsphere with PVA solution to form a paste, then spread the paste onto a flat surface to dry. The dried borosilicate glass microsphere in PVA is then punched with a hole punch for fabrication.

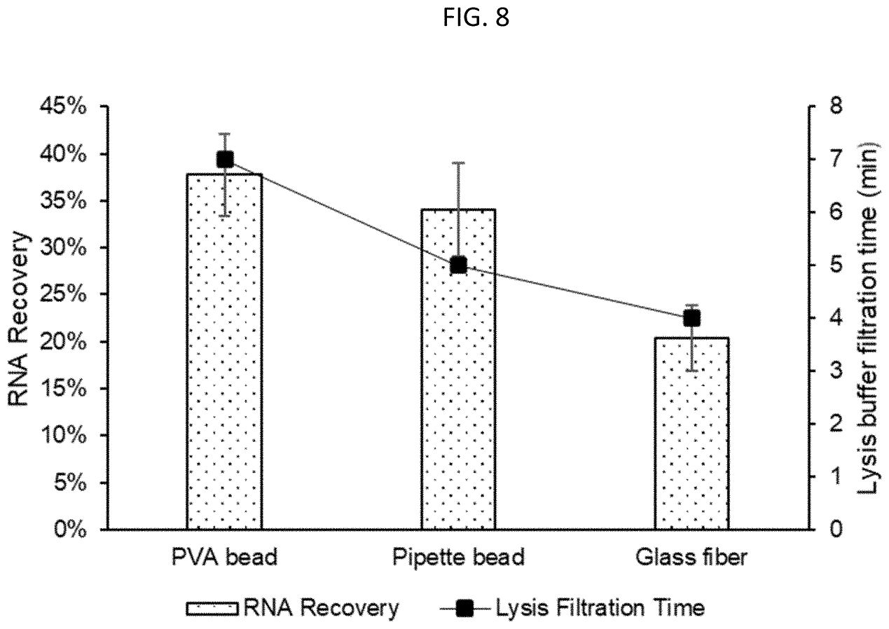

To test RNA recovery, the laminated glass fiber, the laminated glass microspheres prepared using the pipette bead method, and the laminated glass microspheres prepared using the PVA bead method were taped to a 3D-printed funnel using double-sided tape and were placed on top of a wicking pad as shown in FIG. 5. A volume of human total RNA was spiked into 1 ml of a lysis buffer containing guanidium isothiocyanate, ethanol, sodium citrate, EDTA, IGEPAL CA-630, and glycogen. The mixed RNA/lysis buffer was then pipetted into the funnel for the filtration. The time cost was recorded, as shown in FIG. 8. After the filtration, 1 ml of ethanol solution in water was pipetted into the funnel to wash the isolated RNA, after which the RNA was dried at room temperature for 5 min. The elution was performed by pipetting an elution buffer containing sodium citrate and incubating for 5 min. The RNA concentration in the eluate was then quantified using the Invitrogen Qubit 4 Fluorometer to calculate the RNA recovery rate (FIG. 8).

Human DNA isolation using laminated borosilicate glass microspheres. The DNA recovery of various DNA input amounts was compared. Laminated borosilicate glass microspheres were mixed with an equal volume of 7.5% PVA solution, spread onto a petri dish, and dried in an oven. The dried microspheres were punched into biscuits and sandwiched between two pieces of Fusion 5 membrane (Cytiva) to form the laminate microspheres device. The device was prepared using the same PVA bead method described above.

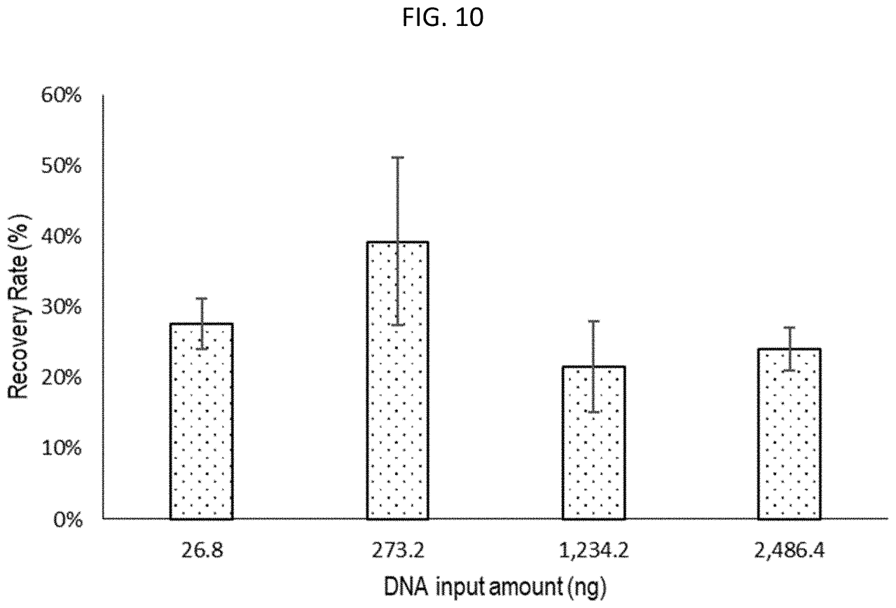

A volume of human genomic DNA was spiked into 1 ml of a lysis buffer containing guanidium isothiocyanate, ethanol, tris, EDTA, IGEPAL CA-630, and glycogen. The mixed DNA/lysis buffer was then pipetted into the funnel for filtration. After filtration, 1 ml of ethanol solution in water was pipetted into the funnel to wash the isolated DNA, and the DNA was then dried at room temperature for 5 min. The elution was performed by adding an elution buffer containing tris and incubating for 5 min. The DNA concentration in the initial input sample and in the eluate were then quantified using the Invitrogen Qubit 4 Fluorometer to calculate the DNA recovery rate (FIG. 10).

1. A point-of-need diagnostic device for detecting a target nucleic acid sequence in a sample, the device comprising a housing with a sample inlet for receiving the sample, wherein the housing contains therein:
(a) a nucleic acid binding stage that is optionally movable comprising a permeable nucleic acid binding substrate and an eluate outlet by which an eluate may exit the binding stage;
(b) a wash reservoir containing a wash buffer;
(c) an elution reservoir containing an elution buffer;
(d) a amplification stage that is optionally movable comprising an eluate inlet for receiving the eluate from the nucleic acid binding stage, at least one reaction chamber comprising a nucleic acid amplification reagent, and an amplicon outlet by which an amplified sample may exit the amplification stage;
(e) a running buffer reservoir containing a running buffer; and
(f) a detection device that provides a readout that indicates whether the target nucleic acid is present in the sample,
wherein the nucleic acid binding stage is configured such that it can be positioned to be in fluid communication with the sample inlet, the wash reservoir, the elution reservoir, and the amplification stage via the eluate outlet;
wherein the amplification stage is configured to be in fluid communication with the running buffer reservoir and the detection device via the amplicon outlet;
wherein in the nucleic acid binding stage is optionally
(i) configured to move in response to a mechanical force or a modulation in a magnetic potential or an electric potential;
(ii) comprises a sponge ramp;
(iii) the permeable nucleic acid binding substrate comprises laminated silica microspheres; or
(iv) any combination thereof;
wherein the device optionally comprises one or more valves for controlling the migration of sample through the device;
wherein the wash reservoir is optionally in fluid communication with a wash sponge or wash hopper;
wherein the elution reservoir is optionally in fluid communication with an elution sponge or elution hopper;
wherein the housing is optionally configured to interface with a lysis cartridge; and
wherein the housing is optionally configured to interface with a controller.

2. A kit comprising the point-of-need diagnostic device of claim 1 and further comprising one or more of a controller, a lysis cartridge, a lysis agent, a collection device, a heating element, or any combination thereof.

3. A method of using the point-of-need diagnostic device of claim 1 to detect the target nucleic acid sequence in the sample, the method comprising:
(a) loading a sample lysate into the diagnostic device via the sample inlet, thereby contacting the sample with nucleic acid binding substrate;
(b) contacting the nucleic acid binding substrate with the wash buffer, wherein the contacting step optionally comprises positioning the nucleic acid binding stage to be in fluid communication with the wash reservoir;
(c) contacting the nucleic acid binding substrate with the elution buffer and eluting the eluate, wherein the contacting step optionally comprises positioning the nucleic acid binding stage to be in fluid communication with the elution reservoir and the amplification stage;
(d) heating the eluate and the nucleic acid amplification reagent in the reaction chamber under conditions sufficient for amplifying the target nucleic acid, thereby preparing the amplified sample;
(e) transferring the amplified sample to the diagnostic device, wherein the transferring step optionally comprises positioning the amplification stage to be in fluid communication with both the running buffer reservoir and the detection device or actuating a valve positioned between the amplification stage and the diagnostic device; and
(f) inspecting the readout provided by the detection device to determine whether the target nucleic acid is present in the sample.

4. The device of claim 1,
wherein the device further comprises a controller configured to interface with the housing and provide the mechanical force or modulate the magnetic potential or the electric potential to move the moveable nucleic acid binding stage into or out of fluid communication with the sample inlet, the wash reservoir, the elution reservoir, and the amplification stage or actuate the one of more valves for controlling the migration of sample through the device at predetermined times,
wherein the controller optionally comprises—
(i) a movable magnet,
(ii) a microcontroller configured to execute a set of instructions for moving the moveable nucleic acid binding stage into or out of fluid communication with the sample inlet, the wash reservoir, the elution reservoir, and the amplification stage or actuating the one or more valves for controlling the migration of sample through the device,
(iii) a heating element,
(iv) a power source,
(v) a readout device, or
(vi) any combination of (i), (ii), (iii), (iv), and (v).

5. The device of claim 1,
wherein the device comprises a lysis cartridge comprising a filter configured to separate particulates from a liquid lysis sample and a sample evacuation device,
wherein—
(i) the filter is optionally a filter membrane or a porous foam filter,
(ii) the filter optionally further comprises a chemical contaminant sequestration material,
(iii) the sample evacuation device is a manually or automatically actuated plunger,
(iv) the sample evacuation device is incapable of evacuating a sample from the lysis cartridge unless a fluid connection is formed between with a sample inlet of a diagnostic device and the lysis cartridge, or
(iv) or any combination of (i), (ii), (iii), and (iv).

6. The device of claim 5, wherein the device further comprises a controller configured to interface with the housing and provide the mechanical force or modulate the magnetic potential or the electric potential to move the moveable nucleic acid binding stage into or out of fluid communication with the sample inlet, the wash reservoir, the elution reservoir, and the amplification stage or actuate the one of more valves for controlling the migration of sample through the device at predetermined times,
wherein the controller optionally comprises—
(i) a movable magnet,
(ii) a microcontroller configured to execute a set of instructions for moving the moveable nucleic acid binding stage into or out of fluid communication with the sample inlet, the wash reservoir, the elution reservoir, and the amplification stage or actuating the one or more valves for controlling the migration of sample through the device,
(iii) a heating element,
(iv) a power source,
(v) a readout device, or
(vi) any combination of (i), (ii), (iii), (iv), and (v).

7. (canceled)

8. (canceled)

9. (canceled)

10. The device of claim 1, wherein the nucleic acid binding substrate comprises a silica membrane and/or silica microspheres.

11. The device of claim 1, wherein the amplification stage comprises a multiplicity of reaction chambers and is configured for:
(a) amplification of two or more different target nucleic acids; or
(b) amplification of one or more target nucleic acids and a positive control.

12. (canceled)

13. The device of claim 1, wherein the nucleic acid amplification reagent comprises:
(a) a DNA polymerase, one or more primers, and nucleoside triphosphates; or
(b) a reverse transcriptase, DNA polymerase, one or more primers, and nucleoside triphosphates.

14. The device of claim 13, wherein the nucleic acid amplification reagent comprises an ICP8 annealase and, optionally, a helicase or a nuclease.

15. (canceled)

16. (canceled)

17. (canceled)

18. (canceled)

19. (canceled)

20. (canceled)

21. (canceled)

22. (canceled)

23. (canceled)

24. The device of claim 1, wherein the detection device is a lateral flow device.

25. (canceled)

26. (canceled)

27. (canceled)

28. (canceled)

29. (canceled)

30. (canceled)

31. (canceled)

32. (canceled)

33. A point-of-need diagnostic device for detecting a target nucleic acid sequence in a sample, the device comprising a housing with a sample inlet for receiving the sample, wherein the housing contains therein,
(a) a reaction chamber comprising a lysate inlet for receiving a lysate from a lysate cartridge, the reaction chamber comprising a nucleic acid amplification reagent, and an amplicon outlet by which an amplified sample may exit the reaction chamber;
(b) a rehydration buffer reservoir containing a rehydration buffer;
(c) a running buffer reservoir containing a running buffer;
(d) a detection device that provides a readout that indicates whether the target nucleic acid is present in the sample; and
(e) a movable wicking channel configured to fluidly connect the reaction chamber and the detection device,
wherein the movable wicking channel is optionally configured to move in response to a modulation in a magnetic potential or an electric potential and
wherein the housing is optionally configured to interface with a controller.

34. The device of claim 33, wherein the device further comprises a controller configured to interface with the housing and modulate the magnetic potential or the electric potential to move the moveable wicking channel into or out of fluid communication with the reaction chamber and the detection device at predetermined times,
wherein the controller optionally comprises—
(i) a movable magnet,
(ii) a microcontroller configured to execute a set of instructions for moving the moveable nucleic acid binding stage into or out of fluid communication with the sample inlet, the wash reservoir, the elution reservoir, and the amplification stage,
(iii) a heating element configured to heat the reaction chamber,
(iv) a heating element configured to heat the lysis cartridge,
(v) a power source,
(vi) a readout device, or
(vii) any combination of (i), (ii), (iii), (iv), (v), and (vi).

35. A method for separating a nucleic acid from a sample, the method comprising:
(a) contacting a sample lysate with a permeable nucleic acid binding substrate, the permeable nucleic acid binding substrate comprising plurality of laminated buoyant, inorganic, nucleic-acid-capture microspheres, thereby adsorbing the nucleic acid; and
(b) contacting the adsorbed nucleic acid with an eluent to form an eluate.

36. The method of claim 35, wherein the plurality of laminated buoyant, inorganic, nucleic-acid-capture microspheres are laminated between fiber pads, optionally wherein the fiber pads comprise paper, silica, or cellulose, or membranes, optionally wherein the membranes are a polymeric membrane.

37. (canceled)

38. The device of claim 1, wherein the device comprises one or more valves for controlling the migration of sample through the device.

39. The device of claim 1, wherein the wash reservoir is in fluid communication with the wash sponge or wash hopper.

40. The device of claim 1, wherein the elution reservoir is in fluid communication with the elution sponge or elution hopper.

41. The device of claim 1, wherein the housing is configured to interface with the lysis cartridge.

42. The device of claim 1, wherein the housing is configured to interface with the controller.
